(19) United States
(12) Patent Application Publication
(10) Pub. No.: US 2020/0214911 A1
(43) Pub. Date: Jul. 9, 2020

(54) ABSORBENT ARTICLE

(71) Applicant: LIVEDO CORPORATION, Ehime (JP)

(72) Inventors: Kenji NAKAOKA, Tokushima (JP); Yuki TAKAHASHI, Tokushima (JP); Masatoshi IKEUCHI, Tokushima (JP)

(73) Assignee: LIVEDO CORPORATION, Ehime (JP)

(21) Appl. No.: 16/647,901

(22) PCT Filed: Sep. 11, 2018
(86) PCT No.: PCT/JP2018/033629

§ 371 Date: Mar. 17, 2020

(30) Foreign Application Priority Data
Sep. 27, 2017 (JP) 2017-186342

(51) Int. Cl. A61F 13/58; A61F 13/62; A61L 15/26; A61L 15/58

(52) U.S. Cl. CPC A61F 13/581; A61L 15/58; A61L 15/26; A61F 13/625

(57) ABSTRACT

An absorbent article (1) comprising a main body part (2) including a top sheet (3), a back sheet (4) and an absorbent core (5) disposed therebetween, and a hook member (11) provided on a non-skin facing side of the main body part (2), wherein the hook member (11) comprises a base part (12) and a plurality of engagement elements (13) projecting from the base part (12), and the base part (12) of the hook member (11) is bonded to the main body part (2) with an adhesive part (14) disposed in a stripe pattern which extends in an extending direction of the hook member (11).

### TECHNICAL FIELD

The present invention relates to an absorbent article such as a disposable diaper, an incontinence pad and a sanitary napkin, and particularly relates to an absorbent article provided with a hook member of a hook-and-loop fastener.

### BACKGROUND ART

Conventionally, absorbent articles provided with a hook member of a hook-and-loop fastener are known. For example, in an incontinence pad or a sanitary napkin, disposing a hook member on a non-skin facing side makes it possible to use by being attached to an underwear such as pants and shorts or a skin facing side of an outer disposable diaper. A hook member is also used in a fastening tape for a tape-type disposable diaper, and when the fastening tape is disposed on both sides of a rear part of the disposable diaper in the width direction and the hook member of the fastening tape is joined to a target tape provided on a front part of the disposable diaper, the disposable diaper can be worn around a wearer's waist.

The hook member provided in the absorbent article can be bonded to the non-skin facing side of the absorbent article or the tape substrate of the fastening tape with an adhesive. For example, Patent Literature 1 discloses that a hook member is bonded to a tape substrate of a fastening tape with an adhesive containing a styrene block polymer as a base polymer.

### CITATION LIST

### Patent Literature

### Patent Literature 1

Japanese Unexamined Laid-open Patent Application Publication No. 2006-223453

### SUMMARY OF INVENTION

### Technical Problem

In the absorbent article provided with a hook member as described above, the absorbent article has increased rigidity at a part where the hook member is disposed, and when wearing such absorbent article, followability to a curved surface of a wearer's body is deteriorated and the wearer tends to feel uncomfortable. The present invention has been achieved in view of the above circumstances, and the object of the present invention is to provide an absorbent article provided with a hook member, that improves followability to a curved surface of a wearer's body at a part where the hook member is disposed.

### Solution to Problem

An absorbent article of the present invention which solves the above problems comprises a main body part including a top sheet, a back sheet and an absorbent core disposed therebetween, and a hook member provided on a non-skin facing side of the main body part, wherein the hook member comprises a base part and a plurality of engagement elements projecting from the base part, and the base part of the hook member is bonded to the main body part with an adhesive part disposed in a stripe pattern which extends in an extending direction of the hook member (hereinafter referred to as an “absorbent article according to a first embodiment”). An absorbent article of the present invention may be also configured so as to comprise a main body part including a top sheet, a back sheet and an absorbent core disposed therebetween, and fastening tapes provided on both sides in a width direction of the main body part, wherein the fastening tape comprises a tape substrate and a hook member provided on the tape substrate, the hook member comprises a base part and a plurality of engagement elements projecting from the base part, and the base part of the hook member is bonded to the tape substrate with an adhesive part disposed in a stripe pattern which extends in a front-rear direction of the absorbent article (hereinafter referred to as an “absorbent article according to a second embodiment”).

It is usual in a hook member provide in an absorbent article that an adhesive part is disposed on a substantially entire surface of the base part to be bonded and fixed to a non-skin facing side of the absorbent article or the tape substrate of the fastening tape; however, in the absorbent article of the present invention, since the base part of the hook member is bonded to the main body part of the absorbent article or the tape substrate of the hook member with the adhesive part disposed in a stripe pattern, the hook member is easily bent at a non-adhesive part formed within the stripe-patterned adhesive part, that is, a non-adhesive part between adjacent strip-shaped adhesive parts. Therefore, followability of the absorbent article to a curved surface of a wearer's body is increased at the part where the hook member is disposed and fittability to a wearer is improved.

In the absorbent article according to the first embodiment, it is preferable that the stripe pattern of the adhesive part is formed by arranging a plurality of thin-linear adhesives, each of which extends in a meandering manner and/or a spiral manner along the extending direction of the hook member, side by side in a width direction of the hook member. In the absorbent article according to the second embodiment, it is preferable that the stripe pattern of the adhesive part is formed by arranging a plurality of thin-linear adhesives, each of which extends in a meandering manner and/or a spiral manner along the front-rear direction of the absorbent article, side by side in a width direction of the absorbent article. When the stripe-patterned adhesive part is formed in this manner, flexibility at the adhesive part can be increased and the followability of the absorbent article to a curved surface of a wearer's body can be further improved at the part where the hook member is disposed.

In the absorbent article according to the first embodiment, it is preferable that the adhesive part is not provided at both end parts, with respect to the width direction of the hook member, of the base part of the hook member, and a non-adhesive part is formed there. In the absorbent article according to the second embodiment, it is preferable that the adhesive part is not provided at both end parts, with respect to the width direction of the absorbent article, of the base part of the hook member, and a non-adhesive part is formed there. By bonding the hook member in this manner, the engaging state of the hook member tends to be stably maintained when the hook member is engaged with another member.

In the absorbent article according to the first embodiment, it is preferable that the non-adhesive part disposed on an outer side in the front-rear or width direction of the absorbent article is formed narrower than the non-adhesive part disposed on an inner side in the front-rear or width direction of the absorbent article. In the absorbent article according to the second embodiment, it is preferable that the non-adhesive part disposed on an outer side in the width direction of the absorbent article is formed narrower than the non-adhesive part disposed on an inner side in the width direction of the absorbent article. By bonding the hook member in this manner, it becomes easy to cancel the engaging state of the hook member when the hook member is detached from another member, meanwhile the engaging state can be stably maintained when the hook member is engaged with another member.

It is preferable that an area of the adhesive part is 50% or more and 85% or less of an area of the hook member. By providing the adhesive part in this manner, the followability of the absorbent article to a curved surface of a wearer's body is easily improved at the part where the hook member is disposed, while securing adhesive force between the hook member and a non-skin facing side of the main body part or the tape substrate.

It is preferable that a peel strength of the hook member is 0.16 N/cm or more and 6.4 N/cm or less. By bonding the hook member to a non-skin facing side of the absorbent article or the tape substrate with such peel strength, the hook member can be stably bonded to the non-skin facing side of the main body part or the tape substrate.

It is preferable that a compression hardness of the main body part or the tape substrate at the part where the hook member is disposed is 0.15 N/cm or more and 0.70 N/cm or less. When the part of the main body part or the tape substrate where the hook member is disposed is formed in this manner, the followability of the main body part or the tape substrate to a curved surface of a wearer's body is easily increased at the part where the hook member is disposed.

It is preferable that the adhesive part is formed from a moisture-curable urethane adhesive. Since the moisture-curable urethane adhesive reacts with moisture in air after applying to accelerate curing, it exhibits sufficient strength even under a humidification condition where the absorbent article is used. In addition, even when the adhesive part is warmed by wearer's body temperature, heat from a heating appliance, or the like in wearing the absorbent article, decrease in peel strength due to softening can be suppressed. The moisture-curable urethane adhesive preferably contains a nonionic surfactant, whereby adhesive strength of the adhesive part can be further increased.

It is preferable that a head part of the engagement elements is oriented in a direction substantially perpendicular to an extending direction of the stripe-patterned adhesive part. By disposing the hook member in this manner, the engaging state where the engagement elements of the hook member are engaged with another member tends to be stably maintained.

### Advantageous Effects of Invention

In the absorbent article of the present invention, since the base part of the hook member is bonded to the main body part of the absorbent article or the tape substrate with the adhesive part disposed in a stripe pattern, the hook member is easily bent at a non-adhesive part formed within the stripe-patterned adhesive part, that is, a non-adhesive part between adjacent strip-shaped adhesive parts. Therefore, the absorbent article of the present invention improves followability to a curved surface of a wearer's body at the part where the hook member is provided and can improve fittability to a wearer.

### BRIEF DESCRIPTION OF THE DRAWINGS

FIG. 1 shows a plan view of an incontinence pad provided with a hook member on a non-skin facing side thereof, as an example of the absorbent article of the present invention, seen from a skin facing side.

FIG. 2 shows a plan view of the absorbent article shown in FIG. 1 seen from the non-skin facing side.

FIG. 3 shows a cross-sectional view along a line III-III of the absorbent article shown in FIGS. 1 and 2.

FIG. 4 shows a cross-sectional view along a line IV-IV of the absorbent article shown in FIG. 2.

FIG. 5 shows a partial enlarged view of the absorbent article shown in FIG. 2 and shows an arrangement pattern of an adhesive part between the hook member and a back sheet in the state where the hook member is removed.

FIG. 6 shows a plan view of a tape-type disposable diaper seen from a skin facing side, as an example of the absorbent article of the present invention.

FIG. 7 shows a cross-sectional view along a line VII-VII of the absorbent article shown in FIG. 6.

FIG. 8 shows a cross-sectional view along a line VIII-VIII of the absorbent article shown in FIG. 6.

FIG. 9 shows a partial enlarged view of a fastening tape of the absorbent article shown in FIG. 6, and shows an arrangement pattern of an adhesive part between the hook member and a tape substrate in the state where the hook member removed.

### DESCRIPTION OF THE PREFERRED EMBODIMENTS

The present invention relates to an absorbent article provided with a hook member. The hook member is a kind of a hook-and-loop fastener, and the hook-and-loop fastener is known by trade names such as “Magic Tape” (registered trademark), “Velcro” (registered trademark), “Quicklon” (registered trademark), and the like. The hook member is configured such that a plurality of engagement elements project from a base part, and is usually used in pairs with a loop member, where the hook member is detachably attached to the loop member or detachably attached to a material functions as a loop member (for example, fabrics such as a nonwoven fabric, a woven fabric and a knitted fabric).

In absorbent articles, the hook member is provided on a non-skin facing side of an incontinence pad or a sanitary napkin, or the hook member is used for a fastening tape of a tape-type disposable diaper. In the former case, the incontinence pad or sanitary napkin provided with the hook member can be attached to an underwear such as pants or shorts by the hook member, or the incontinence pad is disposed on a skin facing side of an outer disposable diaper and can be attached to a skin facing side of the disposable diaper by the hook member. In the latter case, the tape-type disposable diaper is provided with a fastening tape having the hook member on both sides in the width direction of a rear part of the disposable diaper, and by fastening the hook member of the fastening tape to a target tape provide on a front part of the disposable diaper, the disposable diaper can be worn around a wearer's waist.

The present invention relates to an absorbent article provided with a hook member as described above, and the absorbent article of the present invention is explained in detail below with reference to drawings. However, the present invention is not limited to embodiments shown in the drawings.

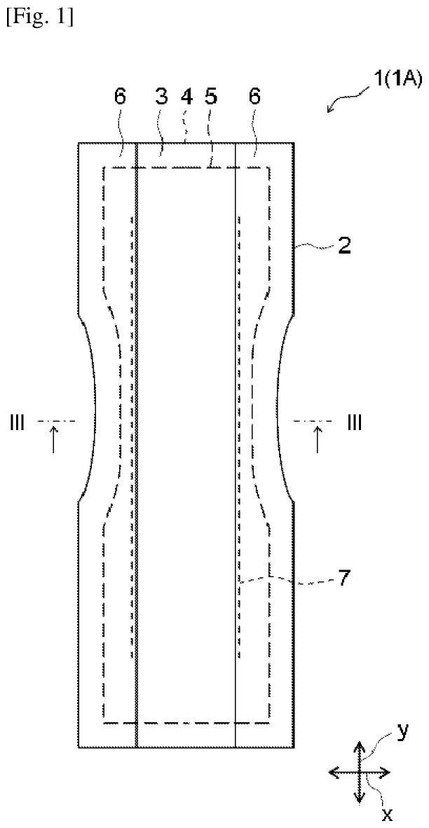

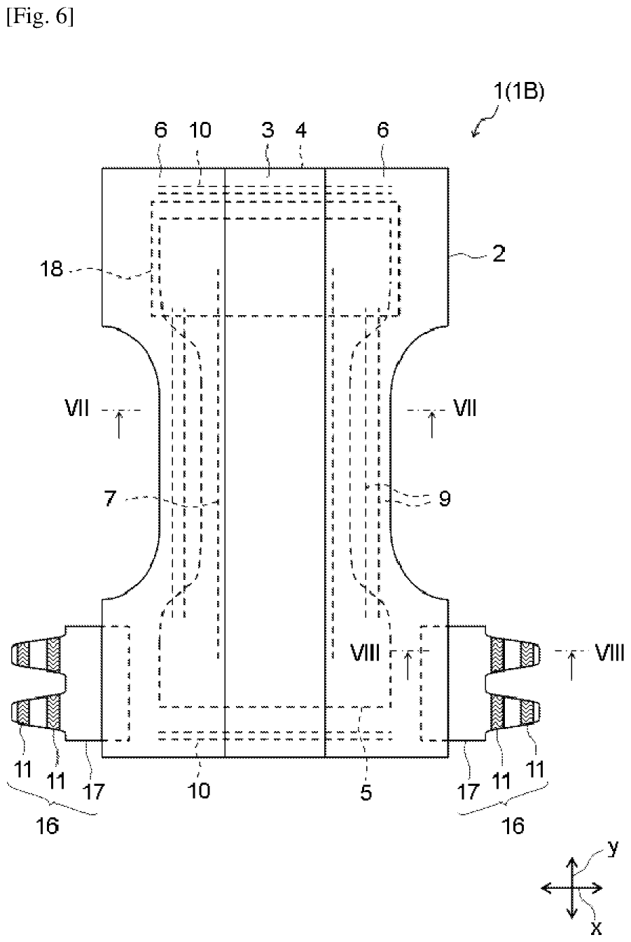

FIGS. 1 to 5 show an absorbent article (an incontinence pad) provided with a hook member on a non-skin facing side thereof as a first embodiment of the absorbent article of the present invention, and FIGS. 6 to 9 show an absorbent article (a tape-type disposable diaper) provided with a fastening tape having a hook member, as a second embodiment of the absorbent article of the present invention. The absorbent articles shown in FIGS. 1 to 5 are indicated with the reference numeral “1” or “1A”, and the absorbent articles shown in FIGS. 6 to 9 are indicated with the reference numeral “1” or “1B”.

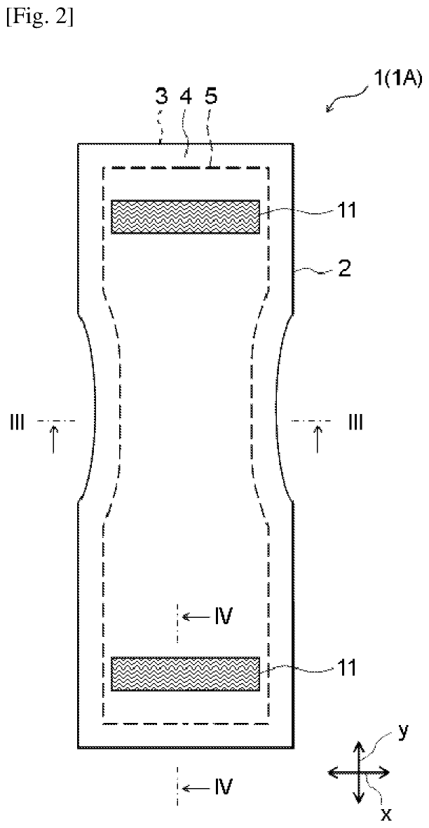

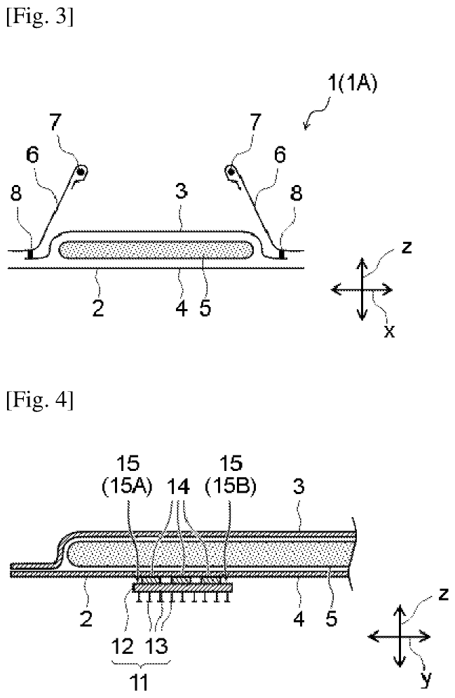

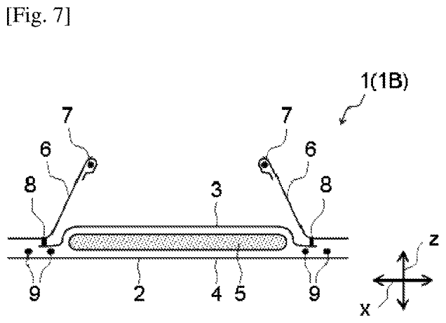

First, configurations other than a hook member of the absorbent article according to the first embodiment and the absorbent article according to the second embodiment are explained with reference to FIGS. 1 to 3, 6, and 7. FIG. 1 shows a plan view of the absorbent article (an incontinence pad) according to the first embodiment as seen from a skin facing side, FIG. 2 shows a plan view of the absorbent article shown in FIG. 1 as seen from a non-skin facing side, FIG. 3 shows a cross-sectional view taken along a line III-III of the absorbent article shown in FIGS. 1 and 2. FIG. 6 shows a plan view of the absorbent article (a tape-type disposable diaper) according to the second embodiment as seen from a skin facing side, and FIG. 7 shows a cross-sectional view taken along a line VII-VII of the absorbent article shown in FIG. 6.

In the drawings, the arrow x represents a width direction of the absorbent article, the arrow y represents a front-rear direction of the absorbent article, and a direction perpendicular to a plane formed by the arrows x and y represents a thickness direction z. The front-rear direction means a direction extending in a front-rear direction at a crotch of a wearer when the wearer wears the absorbent article. The width direction means a direction orthogonal to the front-rear direction on the same plane as the absorbent article, and corresponds to a left-right direction of a wearer when the wearer wears the absorbent article. In addition, a skin facing side means a side facing a wearer's skin and a non-skin facing side means an opposite side of that when the wearer wears the absorbent article.

An absorbent article 1 comprises a main body part 2 including a top sheet 3, a back sheet 4 and an absorbent core 5 disposed therebetween. The main body part 2 constitutes a main part of the absorbent article 1 and functions to receive and absorb urine or the like excreted from a wearer. The main body part 2 has a front part, a rear part and an intermediate part positioned therebetween in a front-rear direction y. In the absorbent article 1B, which is a disposable diaper, the front part corresponds to a part that is applied to an abdomen side of a wearer when wearing the disposable diaper, and the rear part corresponds to a part that is applied to a back side of the wearer when wearing the disposable diaper. The intermediate part corresponds to a part that is positioned between the front part and the rear part and is applied to a wearer's crotch.

The top sheet 3 is a sheet which is disposed on a skin facing side of the absorbent core 5 and is provided so as to face a wearer's skin in wearing the absorbent article. The top sheet 3 is preferably liquid-permeable. As the top sheet 3, a nonwoven fabric made from hydrophilic fibers such as cellulose, rayon and cotton; a nonwoven fabric which is formed from hydrophobic fibers such as polyolefin (e.g., polypropylene and polyethylene), polyester (e.g., PET) and polyamide (e.g., nylon), and in which the hydrophobic fibers are hydrophilized with a surfactant on the surface thereof; or the like can be used, for example. As the top sheet 3, a woven fabric, a knitted fabric, a plastic film having holes may be also used.

The back sheet 4 is a sheet which is disposed on a non-skin facing side of the absorbent core 5 and is provided so as to be positioned on an opposite side of a wearer in wearing the absorbent article. The back sheet 4 is provided so as to constitute at least a part of the surface of a non-skin facing side of the absorbent article. The back sheet 4 is preferably liquid-impermeable. As the back sheet 4, a nonwoven fabric made from hydrophobic fibers such as polyolefin (e.g., polypropylene and polyethylene), polyester (e.g., PET) and polyamide (e.g., nylon); a plastic film; or the like can be used, for example. The back sheet 4 may be composed of a laminate of two or more sheets, and for example, a laminate of a nonwoven fabric and a plastic film may be used. In this case, at least one sheet constituting the laminate may need to be liquid-impermeable, and the size of each sheet constituting the laminate may be the same or different from each other. The meaning of “liquid-impermeable” includes “water-repellent”.

As the nonwoven fabric constituting the top sheet 3 and the back sheet 4, a spunbonded nonwoven fabric, a point-bonded nonwoven fabric, an air-through nonwoven fabric, a meltblown nonwoven fabric, an airlaid nonwoven fabric, a spunlace nonwoven fabric, a tow spread nonwoven fabric, an SMS nonwoven fabric and the like can be used.

The absorbent core 5 is not particularly limited as long as it contains an absorbent material that is able to absorb excrement such as urine. As the absorbent core 5, a shaped product of an absorbent material, which is formed into a predefined shape, may be used, or the shaped product wrapped with a cover sheet such as a paper (e.g., a tissue paper and a thin paper) and a liquid-permeable nonwoven fabric may be used. Examples of the absorbent material include, for example, a hydrophilic fiber such as a cellulose fiber, and an absorbent polymer such as a polyacrylic absorbent polymer, a polyasparaginic absorbent polymer, a cellulosic absorbent polymer and a stark-acrylonitrile absorbent polymer. The absorbent material may include a thermal fusion fiber such as a polyolefin (e.g., polyethylene and polypropylene) fiber, a polyester (e.g., PET) fiber and a polyamide fiber. These thermal fusion fibers may be hydrophilized with a surfactant or the like to increase affinity with urine or the like.

The absorbent material preferably includes a hydrophilic fiber in view of increasing absorption rate of urine or the like. In addition, in view of enhancing absorption capacity, the absorbent material preferably includes an absorbent polymer. Therefore, the absorbent core 5 preferably contains both a hydrophilic fiber (especially a pulp fiber) and an absorbent polymer. In this case, the absorbent material is preferably formed by mixing an absorbent polymer with a hydrophilic fiber assembly, or dispersing an absorbent polymer on a hydrophilic fiber assembly, for example.

The absorbent core 5 may be a sheet-like absorber. Examples of the sheet-like absorber include those that are configured so as to contain an absorbent polymer but not contain a pulp fiber between nonwoven fabrics. Thus formed sheet-like absorber enables having high absorption capacity since it contains an absorbent polymer between nonwoven fabrics. In addition, since the sheet-like absorber does not contain a pulp fiber between nonwoven fabrics, it can be formed thin without being bulky.

For the sheet-shaped absorber, an absorbent fiber may be used as the absorbent material. Also in this case, the sheet-like absorber is formed thin without being bulky. Examples of the absorbent fiber include a fiber having a protonated carboxyl group or a carboxylate group. The absorbent fiber can be obtained by, for example, hydrolyzing an acrylic fiber, thereby converting a nitrile group contained in the acrylic fiber to a carboxylic group. The carboxyl group contained in the absorbent fiber preferably forms an alkaline metal salt or an ammonium salt. The absorbent fiber also can be prepared by immersing a hydrophilic fiber in acrylic acid to deposit acrylic acid on the surface of the fiber.

A shape (planar shape) of the absorbent core 5 is not particularly limited. The shape of the absorbent core 5 may be determined as appropriate according to application, and examples of that include a substantially rectangular shape, an hourglass shape, a center nipped-in gourd shape, a battledore shape and others. In the drawing, the absorbent core 5 is formed in an hourglass shape.

The absorbent article 1 may be provided with side sheets 6 on both sides in the width direction x of the top sheet 3 as a part of the main body part 2. The side sheet 6 is joined to the top sheet 3 at a side joint part 8, and an inner portion from the side joint part 8 in the width direction x is configured to be able to stand up from the top sheet 3, and an outer portion from the side joint part 8 in the width direction x is laminated on the back sheet 4 (see FIGS. 3 and 7). A rising flap is formed when the inner portion of the side sheet 6 in the width direction x stands up, thereby preventing side leakage of urine or the like in the width direction x. For configuring the inner portion of the side sheet 6 in the width direction x to be able to stand up from the top sheet 3, the side sheet 6 is preferably provided with a rising elastic member 7 extending in the front-rear direction y at the inner portion from the side joint part 8 in the width direction x. The side sheet 6 is preferably made of a liquid-impermeable plastic film, a liquid-impermeable nonwoven fabric, or the like.

The absorbent article 1B, which is a tape-type disposable diaper, is preferably provided with leg elastic members 9 extending in the front-rear direction y on both sides in the width direction x. Gathers around legs of the wearer are formed by the leg elastic members 9, that improves fittability around the legs and prevents side leakage.

A waist elastic member 10 extending in the width direction x is preferably provided in an end part in the front-rear direction y of the absorbent article 1B. A waist gather around a wearer's waist is formed by the waist elastic member 10, that prevents leakage of urine or the like from the abdomen or the back.

As each elastic member described above, elastic materials such as a polyurethane thread, a polyurethane film, a natural rubber or the like, that are generally used for absorbent articles such as disposable diapers, can be used. The elastic member is preferably attached in a stretched state with an adhesive such as a hot-melt adhesive. For example, a polyurethane thread having a fineness of 40 dtex to 1,240 dtex is stretched at a ratio of 1.1 to 5.0 times and disposed to be fixed. As the fixing means, a rubber hot-melt adhesive is preferably used. Regarding the above-described ratio, an unexpanded state is defined as 1.0 time.

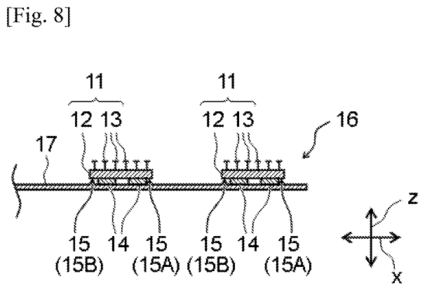

The absorbent article 1 is provided with a hook member 11. In the absorbent article 1A, the hook member 11 is provided on the back sheet 4, that is a non-skin facing side of the main body part 2; and in the absorbent article 1B, a fastening tape 16 in which the hook member 11 is mounted on a tape substrate 17 is provided on both sides in the width direction x of the main body part 2. As shown in FIGS. 4 and 8, the hook member 11 comprises a base part 12 and a plurality of engagement elements 13 projecting from the base part 12, and the base part 12 is bonded and fixed to the non-skin facing side of the main body part 2 or the tape substrate 17 on the opposite surface to the surface provided with the engagement elements 13. FIG. 4 shows a cross-sectional view taken along a line IV-IV of the absorbent article shown in FIG. 2, and FIG. 8 shows a cross-sectional view taken along a line VIII-VIII of the absorbent article shown in FIG. 6. The shape of the engagement element 13 is not particularly limited, and examples thereof include a hook shape, an anchor shape, a mushroom shape, a column shape and others.

In the absorbent article 1A of an incontinence pad, by engaging the engagement elements 13 of the hook member 11 with an underwear such as pants and shorts or a skin facing side of an outer disposable diaper, the absorbent article 1A can be attached to the underwear or the skin facing side of the disposable diaper. In the absorbent article 1A of the incontinence pad, the hook members 11 are often provided on both the front part and the rear part so as to extend in the width direction x, whereby the absorbent article 1A can be stably attached to the underwear or the skin facing side of the disposable diaper with the hook member 11 while reducing an installation area of the hook member 11 (see FIG. 2). Though it is not shown in the drawing, in some case that the hook member 11 is provided on the non-skin facing side of the absorbent article 1, the hook member 11 may be provided so as to extend in the front-rear direction y of the absorbent article 1. For example, in an absorbent article having wing parts, that is configured so that both side parts in the width direction of the main body part extends outward in the width direction, the hook member may be provided on the non-skin facing side of the wing part, which is a portion of the main body part, so as to extend in the front-rear direction.

In the absorbent article 1B of a tape-type disposable diaper, the fastening tapes 16 are provided on both sides in the width direction x of the rear part as shown in FIG. 6, and by engaging the engagement elements 13 of the hook member 11 provided on the fastening tape 11 with a target tape 18 provided on the non-skin facing side of the front part of the absorbent article 1B, the absorbent article 1B can be formed into a pants shape and can be worn. The target tape 18 can be composed of a loop member of a hook-and-loop fastener. In the absorbent article 1B of the tape-type disposable diaper, the hook member 11 is usually provided so as to extend in the front-rear direction y of the absorbent article 1B, whereby the fastening tape 16 can be stably fastened to the target tape 18 when wearing the absorbent article 1B.

By the way, in the absorbent article 1 provided with the hook member 11 as described above, it is usual for the hook member 11 that the substantially entire surface of the base part 12 is bonded and fixed to the back sheet 4 or the tape substrate 17 of the fastening tape 16. Specifically, in the hook member 11, an adhesive is applied to substantially the entire surface of the base part 12, which is opposite surface to the surface on which the engagement elements 13 is provided, and the substantially entire surface of the base part 12 is bonded to the back sheet 4 or the tape substrate 17 of the fastening tape 16. Therefore, the absorbent article 1 comes to have increased rigidity at the part where the hook member 11 is attached, and when wearing such absorbent article, followability to a curved surface of a wearer's body is decreased at the part where the hook member 11 is attached and the wearer tends to feel uncomfortable.

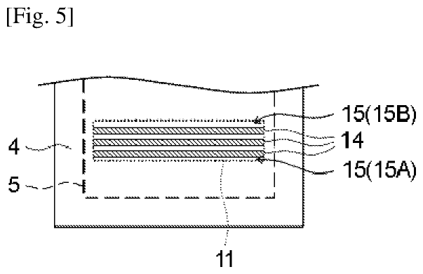

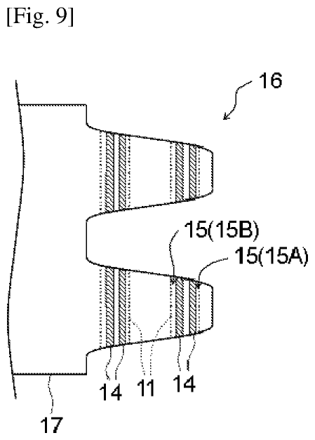

Therefore, in the absorbent article 1, the base part 12 of the hook member 11 is bonded to the main body part 2 or the tape substrate 17 of the fastening tape 16 with an adhesive part 14 disposed in a stripe pattern. This is explained with reference to FIGS. 5 and 9 in addition to FIGS. 4 and 8. FIG. 5 shows a partial enlarged view of the absorbent article shown in FIG. 2 and shows an arrangement pattern of the adhesive part between the hook member and the back sheet in the state where the hook member is removed from the absorbent article. FIG. 9 shows a partial enlarged view of the fastening tape of the absorbent article shown in FIG. 6 and shows an arrangement pattern of the adhesive part between the hook member and the tape substrate in the state where the hook member removed. In FIGS. 5 and 9, the outline of the part where the hook member is provided is indicated by a dotted line.

In the absorbent article 1A, the base part 12 of the hook member 11 is bonded to the main body part 2 with an adhesive part 14 disposed in a stripe pattern which extends in an extending direction of the hook member 11. By bonding the hook member 11 to the main body part 2 in this manner, followability of the absorbent article 1A to a curved surface of a wearer's body is improved at the part where the hook member 11 is disposed. Thus, since the hook member 11 is easily bent at a non-adhesive part formed within the stripe-patterned adhesive part 14 (that is, a non-adhesive part between adjacent strip-shaped adhesive parts 14), fittability to a wearer's crotch is improved at the part where the hook member 11 is disposed when the absorbent article 1A is attached to an underwear or a skin facing side of the disposable diaper and is worn. Here, the hook member 11 is usually formed such that both side edges along the extending direction of the hook member 11 (that is, both side edges with respect to a width direction of the hook member 11) have a straight line shape that is substantially parallel to the extending direction of the hook member 11. The extending direction of the hook member 11 usually substantially coincides with a longitudinal direction of the hook member 11.

In the absorbent article 1B, the base part 12 of the hook member 11 is bonded to the tape substrate 17 with the adhesive part 14 disposed in a stripe pattern which extends in the front-rear direction y of the absorbent article 1B. By bonding the hook member 11 to the tape substrate 17 in this manner, followability of the fastening tape 16 to a curved surface of a wearer's body is improved when the absorbent article 1B is worn. Specifically, when the absorbent article 1B is worn around a wearer's waist, the fastening tape 16 is curved along a wearer's waist, and in the absorbent article 1B, since the stripe-patterned adhesive part 14 is provided so as to extend in the front-rear direction y of the absorbent article 1B, the hook member 11 is easily bent in the width direction x of the absorbent article 1B at a non-adhesive part formed within the stripe-patterned adhesive part 14 (that is, a non-adhesive part between adjacent strip-shaped adhesive parts 14). Therefore in the absorbent article 1B, fittability of the fastening tape 16 around a wearer's waist can be improved at the part where the hook member 11 is provided.

In the absorbent article 1A, it is preferable that the adhesive part 14 is not provided at both end parts, with respect to the width direction of the hook member 11, of the base part 12 of the hook member 11 and non-adhesive parts 15 are formed there. In the absorbent article 1B, it is preferable that the adhesive part 14 is not provided at both end parts, with respect to the width direction x of the absorbent article 1B, of the base part 12 of the hook member 11 and non-adhesive parts 15 are formed there. Hereinafter, the non-adhesive part 15 is referred to as an edge non-adhesive part 15. By providing this edge non-adhesive part 15 on the base part 12 of the hook member 11, it becomes easy to stably maintain the engaging state where the engagement element 13 of the hook member 11 is engaged with another member. In the absorbent article 1A, when the base part 12 of the hook member 11 is provided with the edge non-adhesive parts 15 at both end parts in the width direction of the hook member 11, the base part 12 of the hook member 11 is difficult to be peeled off starting from the both end parts thereof even in the case where the absorbent article 1A is worn by attaching to an underwear or a skin facing side of a disposable diaper and a force that shifts the absorbent article 1A with respect to the underwear or the disposable diaper is applied to the absorbent article 1A. In the absorbent article 1B, when the base part 12 of the hook member 11 is provided with the edge non-adhesive parts 15 at both end parts in the width direction x of the absorbent article 1B, the base part 12 of the hook member 11 is difficult to be peeled off starting from the both end parts thereof even in the case where a force that shifts the fastening tape 16 with respect to the target tape is applied to the fastening tape 16. As a result, the hook member 11 can be stably engaged with another member.

Regarding the edge non-adhesive part 15, it is preferable that the non-adhesive part 15A disposed on an outer side in the front-rear direction y or the width direction x of the absorbent article 1 is formed narrower than the non-adhesive part 15B disposed on an inner side in that direction. Specifically in the absorbent article 1B, the edge non-adhesive parts 15 are preferably formed so that the edge non-adhesive part 15A disposed on an outer side in the width direction x of the absorbent article 1 is narrower than the edge non-adhesive part 15B disposed on an inner side in that direction. In the absorbent article 1A, in the case where the hook member 11 is disposed so as to extend in the width direction x of the absorbent article 1A as shown in FIGS. 1 and 2, the edge non-adhesive parts 15 are preferably formed so that the edge non-adhesive part 15A disposed on an outer side in the front-rear direction y of the absorbent article 1 is narrower than the edge non-adhesive part 15B disposed on an inner side in that direction. By forming the edge non-adhesive parts 15 in this manner, it becomes easy to cancel the engaging state of the hook member 11 when the hook member 11 is detached from another member, meanwhile the engaging state of the hook member 11 is stably maintained when the hook member 11 is engaged with another member. Specifically, in the absorbent article 1A, by forming a width of the edge non-adhesive part 15A on the side close to an edge of the absorbent article 1A narrower, a force by which the absorbent article 1A is gripped and pulled is easily transmitted to the hook member 11 and the engagement of the hook member 11 with another member is easily released when the absorbent article 1A is detached from an underwear or a disposable diaper. In the absorbent article 1B, by forming a width of the edge non-adhesive part 15A on the outer side in the width direction x narrower, a force by which the fastening tape 16 is gripped and pulled is easily transmitted to the hook member 11 and the engagement of the hook member 11 with the target tape 18 is easily released when the fastening tape 16 is detached from the target tape 18.

The stripe-patterned adhesive part 14 is formed by arranging a plurality of strip-shaped adhesive parts in parallel to each other, and the number of the strip-shaped adhesive part is not particularly limited as long as it is two or more. The width of the strip-shaped adhesive part is, for example, preferably 2 mm or more, more preferably 3 mm or more, and preferably 10 mm or less, more preferably 8 mm or less. The non-adhesive part is formed between the adjacent adhesive parts, and provided that this non-adhesive part is referred to as an intermediate non-adhesive part, the width of the intermediate non-adhesive part is, for example, preferably 0.5 mm or more, more preferably 1 mm or more, and preferably 5 mm or less, more preferably 4 mm or less. The width of the edge non-adhesive part 15 described above is, for example, preferably 0.5 mm or more, more preferably 1 mm or more, and preferably 5 mm or less, more preferably 4 mm or less. The width of the hook member 11 is preferably 5 mm or more, more preferably 8 mm or more, even more preferably 10 mm or more, and preferably 40 mm or less, more preferably 30 mm or less, even more preferably 25 mm or less.

The width of each strip-shaped adhesive part constituting the stripe-patterned adhesive part 14 may be the same as or different from each other. For example, the adhesive part 14 may be formed so that the adhesive part disposed on the outer side in the front-rear direction y or the width direction x of the absorbent article 1 is narrower than the adhesive part disposed on the inner side in that direction. By providing the adhesive part in this manner, the hook member 11 comes to have increased rigidity on the outer side in the front-rear direction y or the width direction x of the absorbent article 1, and the engaging state of the hook member 11 tends to be stably maintained when the engagement elements 13 thereof engage with another member. For example, in the absorbent article 1A, even when the end part of the absorbent article 1A in the front-rear direction y or the width direction x is turned up in the state where the absorbent article 1A is attached to an underwear or a skin facing side of a disposable diaper, release of the engagement of the hook member 11 with the underwear or the disposable diaper is less likely to occur in accordance with that. In the absorbent article 1B, even when the end part of the fastening tape 16 in the width direction x is turned up in the state where the fastening tape 16 is attached to the target tape 18, release of the engagement of the hook member 11 with the target tape is less likely to occur in accordance with that.

Each of the strip-shaped adhesive parts constituting the stripe-patterned adhesive part 14 may be formed by solid coating or may be formed by applying a thin-linear adhesive in a predetermined or random pattern in strip-shaped areas. In particular, from the viewpoint of increasing flexibility at the adhesive part 14, each of the strip-shaped adhesive parts is preferably formed in the latter manner, whereby the followability of the absorbent article 1 to a curved surface of a wearer's body can be further improved at the part where the hook member 11 is disposed. In this case, the stripe-patterned adhesive part 14 is preferably formed as follows. In the absorbent article 1A, the stripe-patterned adhesive part 14 is preferably formed by arranging a plurality of thin-linear adhesives, each of which extends in a meandering manner and/or a spiral manner along the extending direction of the hook member 11, side by side in the width direction of the hook member 11. In the absorbent article 1B, the stripe-patterned adhesive part 14 is preferably formed by arranging a plurality of thin-linear adhesives, each of which extends in a meandering manner and/or a spiral manner along the front-rear direction y, side by side in the width direction x. By applying the thin-linear adhesives in this manner, the adhesive part 14 is easily formed in a stripe pattern.

The adhesive part 14 is preferably formed from a hot-melt adhesive. Thereby, the adhesive can be applied in a finer pattern. As the hot-melt adhesive, a rubber adhesive such as natural rubber, butyl rubber and polyisoprene; styrene elastomer such as styrene-isoprene-styrene block copolymer (SIS), styrene-butadiene-styrene block copolymer (SBS), styrene-ethylene-butadiene-styrene block copolymer (SEBS) and styrene-ethylene-propylene-styrene block copolymer (SEPS); or a reactive urethane adhesive is preferably used as a base polymer, The base polymer may be blended with ethylene-vinyl acetate copolymer (EVA); polyester; acrylic elastomer; polyolefin elastomer or the like to adjust tackiness or fluidity. It is also preferable to use an amorphous polyalphaolefin (APAO) such as ethylene-propylene copolymer as a kind of polyolefin elastomer.

In view of increasing adhesive strength of the adhesive part 14, it is also preferable that the adhesive part 14 is formed from a moisture-curable urethane adhesive. Since the moisture-curable urethane adhesive contains isocyanate group-terminated urethane prepolymer and reacts with moisture in air after applying to accelerate curing, it exhibits sufficient strength even under humidification conditions where the absorbent article is used. In addition, even when the adhesive part 14 is warmed by wearer's body temperature, heat from a heating appliance, or the like in wearing the absorbent article, decrease in peel strength due to softening can be suppressed.

Urethane prepolymer can be obtained by reacting polyisocyanate and polyol. As the polyol, polyether polyol such as polyethylene glycol, polypropylene glycol and polytetramethylene ether glycol; polyester polyol such as poly(ethylene adipate), poly(butylene adipate), poly(hexamethylene adipate), poly(butylene azelate), poly(butylene sebacate) and polycaprolactone; polycarbonate polyol or the like can be used. As the polyisosyanate, aromatic diisocyanate such as 2,4- and/or 2,6-tolylene diisocyanate (TDI), 2,4′- and/or 4,4′-diphenylmethane diisocyanate (MDI), 1,5-naphthalene diisocyanate (NDI), tolidine diisocyanate (TODI) and xylene diisocyanate (XDI); or aliphatic diisocyanate such as hexamethylene diisocyanate (HDI), hydrogenated XDI (H₆XDI), hydrogenated MDI (H₁₂MDI) and isophorone diisocyanate (IPDI) can be used.

The moisture-curable urethane adhesive is preferably a hot-melt adhesive. Thereby, it becomes easy to apply the moisture-curable urethane adhesive in a finer pattern. The melting temperature of the moisture-curable urethane adhesive is, for example, preferably 70° C. to 200° C. and more preferably 80° C. to 160° C.

The adhesive forming the adhesive part 14 may contain additives such as a plasticizer, a tackifier, various fillers, a pigment, a wax, a viscosity modifier, a moisture remover, a storage stabilizer and an antioxidant as necessary. In the case of using a moisture-curable urethane adhesive as the adhesive, it is preferable that a nonionic surfactant is preferably contained as an additive, and as a result, adhesive strength of the adhesive part 14 can be increased. Examples of the nonionic surfactant include polyhydric alcohol fatty acid esters such as sucrose fatty acid esters, glycerin fatty acid esters and sorbitan fatty acid esters; polyoxyalkylene ethers such as polyoxyalkylene alkyl ethers and polyoxyalkylene alkyl phenyl ethers; and oxyalkylene fatty acid esters. For these surfactant, one kind thereof may be used or two or more kinds thereof may be used in combination. Among them, from the viewpoint that the moisture-curable urethane adhesive suppresses generation of bubbles in reacting with moisture in air and is excellent in permeability of the adhesive to the main body part 2 or the tape substrate 17, polyoxyalkylene ethers are preferably used.

The area of the adhesive part 14 is preferably 50% or more of the area of the hook member 11, more preferably 60% or more of that, and preferably 85% or less of that, more preferably 75% or less of that. By providing the adhesive part 14 in this manner, the followability to a curved surface of a wearer's body is easily improved at the part where the hook member 11 is disposed, while securing adhesive force between the base part 12 of the hook member 11 and a non-skin facing side of the main body part 2 or the tape substrate 17. Here, the sum total of the area of the adhesive part and the area of the non-adhesive part (including the intermediate non-adhesive part and the edge non-adhesive part) is 100%.

A peel strength of the hook member 11 is preferably 0.16 N/cm or more, more preferably 0.32 N/cm or more and even more preferably 0.64 N/cm or more. By bonding the base part 12 of the hook member 11 to the non-skin facing side of the main body part 2 (for example, the back sheet 4) or the tape substrate 17 with such peel strength, the base part 12 of the hook member 11 can be stably bonded to the non-skin facing side of the main body part 2 or the tape substrate 17. The upper limit of the peel strength of the hook member 11 is not particularly limited, but is preferably 6.4 N/cm or less, more preferably 4.8 N/cm or less and even more preferably 3.2 N/cm or less. The peel strength of the hook member is measured in accordance with JIS K 6854-3 (1999) “Testing Methods For Peel Strength of Adhesives, Part 3: T-Peer Method”, and a test piece for the peel strength measurement is cut out from an absorbent article such that the stripe-patterned adhesive part extends in a longitudinal direction of the test piece. A width of the test piece corresponds to the width of the hook member, and a peel strength value obtained by the test is divided by the width of the test piece, thereby obtaining the value per 1 cm width.

A compression hardness of the main body part 2 or the tape substrate 17 at the part where the hook member 11 is disposed is preferably 0.70 N/cm or less, more preferably 0.55 N/cm or less and even more preferably 0.40 N/cm or less. When the part of the main body part 2 or the tape substrate 17 where the hook member 11 is disposed is formed in this manner, the followability of the main body part 2 or the tape substrate 17 to a curved surface of a wearer's body is easily improved at the part where the hook member 11 is disposed. The lower limit of the compression hardness of the main body part 2 or the tape substrate 17 at the part where the hook member 11 is disposed is not particularly limited, but is preferably 0.15 N/cm or more, more preferably 0.20 N/cm or more and even more preferably 0.25 N/cm or more, from the viewpoint of securing a certain degree of stiffness in that part.

The compression hardness is obtained by the following method. The test piece used for measuring the compression hardness is cut out at a width of 20 mm such that the stripe-patterned adhesive part extends in a width direction of the test piece from the part of the main body part or the tape substrate where the hook member is disposed. At this time, only the sheet member to which the hook member is attached (for example, the back sheet) is used for the test as the main body part. Using a tensile and compression testing machine (for example, Tensilon (registered trademark) RTG-1210, available from A & D Company, Ltd.), the cut out test piece is set between chucks so that the distance between the chucks is 10 mm (where the chuck holds the hook member and the main body part or the tape substrate so that the hook member exists between the chucks), and then is compressed at a compression speed of 50 mm/min until the distance between the chucks is 5 mm. The compression stress per 1 cm width of the test piece at that time (that is, the value obtained by dividing the value of the stress measured by the tensile and compression testing machine by 2) is determined as the compression hardness.

The hook member 11 is preferably disposed on the main body part 2 or the tape substrate 17 as follows. That is, the hook member 11 is preferably disposed on the main body part 2 or the tape substrate 17 so that a head part of the engagement elements 13 of the hook member 11 is oriented in a direction substantially perpendicular to the extending direction of the stripe-patterned adhesive part 14. More specifically, it is preferable that the longitudinal direction of the head part of the engagement elements 13 of the hook member 11 is substantially perpendicular to the extending direction of the stripe-patterned adhesive part 14 when the hook member 11 is viewed in plan. By disposing the hook member 11 in this manner, the engaging state where the engagement elements 13 of the hook member 11 are engaged with another member tends to be stably maintained. For example, in the case where the absorbent article 1A is worn by being attached to an underwear or a skin facing side of a disposable diaper, when a force for shifting the hook member 11 in the width direction thereof is applied, the hook member 11 is easily peeled off from the underwear or the disposable diaper. However, by forming the engagement elements 13 of the hook member 11 as described above, the engagement elements 13 of the hook member 11 are hooked to the underwear or the disposable diaper even when the force for shifting the hook member 11 in the width direction thereof is applied, and as a result, the engaging state tends to be stably maintained. In the absorbent article 1B, even when a force for shifting the fastening tape 16 in the width direction x with respect to the target tape is applied, the engagement elements 13 of the hook member 11 are hooked on the target tape, and the engaging state tends to be stably maintained.

The orientation direction of the head part of the engagement element 13 is explained as follows. In the case where the shape of the engagement element 13 is a hook shape, the head part of the engagement element 13 is bent in one direction, and this bending direction is the orientation direction of the head part. In the case where the shape of the engagement element 13 is an anchor shape, a crossbar is provided at the top of the engagement element 13, and the extending direction of the crossbar is the orientation direction of the head part. In the case where the shape of the engagement element 13 is a mushroom shape, a cap is provided at the top of the engagement element 13, and this cap has anisotropy so as to have a minor axis direction and a major axis direction. The major axis direction of this cap is the orientation direction of the head part

This application claims priority to Japanese Patent Application No. 2017-186342, filed on Sep. 27, 2017. All of the contents of the Japanese Patent Application No. 2017-186342, filed on Sep. 27, 2017, are incorporated by reference herein.

### REFERENCE SIGNS LIST

1: an absorbent article

2: a main body part

3: a top sheet

4: a back sheet

5: an absorbent core

6: a side sheet

7: a rising elastic member

8: a side joint part

9: a leg elastic member

10: a waist elastic member

11: a hook member

12: a substrate

13: an engagement element

14: an adhesive part

15: a non-adhesive part (at an end side part)

16: a fastening tape

17: a tape substrate

18: a target tape

1. An absorbent article comprising a main body part including a top sheet, a back sheet and an absorbent core disposed therebetween, and a hook member provided on a non-skin facing side of the main body part, wherein
the hook member comprises a base part and a plurality of engagement elements projecting from the base part, and
the base part of the hook member is bonded to the main body part with an adhesive part disposed in a stripe pattern which extends in an extending direction of the hook member.

2. An absorbent article comprising a main body part including a top sheet, a back sheet and an absorbent core disposed therebetween, and fastening tapes provided on both sides in a width direction of the main body part, wherein
the fastening tape comprises a tape substrate and a hook member provided on the tape substrate,
the hook member comprises a base part and a plurality of engagement elements projecting from the base part, and
the base part of the hook member is bonded to the tape substrate with an adhesive part disposed in a stripe pattern which extends in a front-rear direction of the absorbent article.

3. The absorbent article according to claim 1, wherein
the stripe pattern of the adhesive part is formed by arranging a plurality of thin-linear adhesives, each of which extends in a meandering manner and/or a spiral manner along the extending direction of the hook member, side by side in a width direction of the hook member.

4. The absorbent article according to claim 2, wherein
the stripe pattern of the adhesive part is formed by arranging a plurality of thin-linear adhesives, each of which extends in a meandering manner and/or a spiral manner along the front-rear direction of the absorbent article, side by side in a width direction of the absorbent article.

5. The absorbent article according to claim 1, wherein
the adhesive part is not provided at both end parts, with respect to the width direction of the hook member, of the base part of the hook member, and a non-adhesive part is formed there.

6. The absorbent article according to claim 2, wherein
the adhesive part is not provided at both end parts, with respect to the width direction of the absorbent article, of the base part of the hook member, and a non-adhesive part is formed there.

7. The absorbent article according to claim 5, wherein
the non-adhesive part disposed on an outer side in the front-rear or width direction of the absorbent article is formed narrower than the non-adhesive part disposed on an inner side in the front-rear or width direction of the absorbent article.

8. The absorbent article according to claim 6, wherein
the non-adhesive part disposed on an outer side in the width direction of the absorbent article is formed narrower than the non-adhesive part disposed on an inner side in the width direction of the absorbent article.

9. The absorbent article according to claim 1, wherein
an area of the adhesive part is 50% or more and 85% or less of an area of the hook member.

10. The absorbent article according to claim 1, wherein
a peel strength of the hook member is 0.16 N/cm or more and 6.4 N/cm or less.

11. The absorbent article according to claim 1, wherein
a compression hardness of the main body part or the tape substrate at a part where the hook member is disposed is 0.15 N/cm or more and 0.70 N/cm or less.

12. The absorbent article according to claim 1, wherein the adhesive part is formed from a moisture-curable urethane adhesive.

13. The absorbent article according to claim 12, wherein the adhesive contains a nonionic surfactant.

14. (canceled)

15. The absorbent article according to claim 2, wherein
a head part of the engagement elements is oriented in a direction substantially perpendicular to an extending direction of the stripe-patterned adhesive part.

16. The absorbent article according to claim 2, wherein
an area of the adhesive part is 50% or more and 85% or less of an area of the hook member.

17. The absorbent article according to claim 2, wherein
a peel strength of the hook member is 0.16 N/cm or more and 6.4 N/cm or less.

18. The absorbent article according to claim 2, wherein
a compression hardness of the main body part or the tape substrate at a part where the hook member is disposed is 0.15 N/cm or more and 0.70 N/cm or less.

19. The absorbent article according to claim 2, wherein the adhesive part is formed from a moisture-curable urethane adhesive.

20. The absorbent article according to claim 19, wherein the adhesive contains a nonionic surfactant.

21. (canceled)
